(19) United States
(12) Patent Application Publication
(10) Pub. No.: US 2026/0061607 A1
(43) Pub. Date: Mar. 5, 2026

(54) METHOD AND SYSTEM FOR AUTOMATED AND SEMI-AUTOMATED ULTRASOUND SCANNING

(71) Applicant: The Governors of the University of Alberta, Edmonton (CA)

(72) Inventors: Kumaradevan Punithakumar, Edmonton (CA); Harald Hans Becher, Edmonton (CA); Jacob Jaremko, Edmonton (CA); Michelle Lisa Noga, Edmonton (CA); Pierre Boulanger, Edmonton (CA); Nilanjan Ray, St Albert (CA); Ahmed Sharif Ahmed, Edmonton (CA); Jonathan David Windram, Edmonton (CA)

(21) Appl. No.: 19/316,662

(22) Filed: Sep. 2, 2025

Related U.S. Application Data
(60) Provisional application No. 63/689,502, filed on Aug. 30, 2024.

(51) Int. Cl. B25J 9/16; A61B 8/00; A61B 8/08; B25J 13/08; B25J 15/00; G06T 7/30; G06T 7/70

(52) U.S. Cl. CPC B25J 9/1633; A61B 8/0883; A61B 8/4218; A61B 8/54; B25J 9/1684; B25J 13/085; B25J 15/0019; G06T 7/30; G06T 7/70; G06T 2207/10132; G06T 2207/30004

(57) ABSTRACT

Disclosed examples generally relate to a robot arm-based ultrasound or echocardiography scanning system configured to mitigate limitations associated with conventional manual scanning, including sonographer strain, restricted field-of-view, and low signal-to-noise image quality. In such examples, the patient is scanned using a transducer mounted on and manipulated by a robot arm. The cardiac structures of subjects are imaged from multiple positions using a 2D and 3D echocardiography scanning system.

### CROSS-REFERENCE TO RELATED APPLICATION

This application claims the priority benefit of U.S. Provisional Patent Application No. 63/689,502, filed on Aug. 30, 2024, the entire contents of which are incorporated herein by reference.

### FIELD

The present disclosure generally relates to ultrasounds and echocardiograms, and more particularly, to a method and system for automated or semi-automated ultrasound or echocardiography scanning.

### BACKGROUND

Echocardiography is widely used worldwide to non-invasively image the anatomy and function of the heart to diagnose cardiac disease. It uses ultrasound waves to generate images of the heart and does not involve ionizing radiation, making it one of the safest modalities to scan the heart. In addition, it is portable and inexpensive compared to other modalities available for scanning the heart, such as magnetic resonance imaging or computed tomography.

Two-dimensional (2D) echocardiography allows for obtaining a slice image of the heart, and the corresponding field-of-view is limited only to a 2D plane. In contrast, three-dimensional (3D) echocardiography allows for imaging of the anatomy and function of the heart in 3D space and significantly improves the field of view of the modality. Although this approach leads to either reduction in image quality or frame rate when the heart is scanned within one cardiac cycle, multi-beat acquisitions vastly improve image quality and frame rate. In multi-beat acquisitions, a sub-volume of the heart is imaged in each heartbeat.

Despite the improvement in the field of view, 3D echocardiography cannot image the entire heart in a single scan in most adult patients. The angle of the incident of the ultrasound waves should ideally be perpendicular to the organ boundary to obtain an optimal quality image, and echocardiography suffers from occasional signal dropout when the angle of the incident is small. One option to overcome these limitations is to scan the heart from different positions. Apical and parasternal views are two of the commonly used views in clinics to scan the heart to get different parts imaged adequately.

### SUMMARY OF VARIOUS EMBODIMENTS

Disclosed examples generally relate to a robot arm-based ultrasound or echocardiography system to mitigate problems such as sonographer strain, field-of-view, low signal-to-noise image quality, by moving the transducer attached to the arm to perform ultrasound scanning. In one embodiment of a disclosed method, the cardiac structures of human participants can be imaged from multiple positions using a 2D or 3D echocardiography scanning system. The system can also be utilized to identify optimal positions of the transducer during 2D or 3D echocardiography procedures.

In one aspect, disclosed is a collaborative robot system for ultrasound scanning, comprising:
a robot arm;
an ultrasound transducer coupled to an end of the robot arm;
a controller for controlling movement of the robot arm;
at least one processor configured for:
   determining a goal view;
   selecting a trained navigation model associated with the goal view;
   applying the selected navigation model to generate movement coordinates for translating and orienting the robot arm to the goal view;
   applying a contact maintenance model to generate an orientation command for the robot arm;
   applying a safe actuation model based on the outputs of the navigation model and contact maintenance model; and
   operating the robot arm based on torque and force outputs generated by the safe actuation model.

In preferred embodiments, the system is configured for echocardiography scanning, and the at least one processor comprises an image analysis module configured for aligning images generated from scans from different goal views. The robot arm may comprise force and/or torque sensors which modulate the navigation model, the contact maintenance model, and/or the safe actuation model to regulate contact of the transducer with a patient.

In preferred embodiments, the contact maintenance model is configured to maintain a consistent orientation and constant contact force with the patient once the transducer is correctly positioned on the patient for the selected goal view.

In another aspect, disclosed is a method of controlling a robot arm for ultrasound scanning of a patient, using an ultrasound transducer coupled to an end of the robot arm, comprising the steps of:
determining a goal view;
selecting a trained navigation model associated with the goal view;
applying the selected navigation model to generate movement coordinates for translating the robot arm to the goal view;
applying a contact maintenance model to generate a movement command and/or an orientation command for the robot arm;
applying a safe actuation model based on the outputs of the navigation model and contact maintenance model; and
operating the robot arm based on torque and force outputs generated by the safe actuation model.

In preferred embodiments, the method comprises acquiring images from echocardiography scanning from different goal views, and using an image analysis module to align those images.

In some embodiments, the disclosed system or method may comprise any combination or sub-combination of features, elements, or steps disclosed or referred to herein. The combination or sub-combination may include or omit any preferred or alternative feature, element or step.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A shows the overall system arrangement showing the components of one embodiment the robot multi-view echocardiography scanning system.

FIG. 1B is another image of the components of the robot echocardiography scanning system. The movements of the robot arm are replayed as animations with the corresponding force values in the x, y, and z directions as shown in the graphs on the right-hand side of the image.

FIG. 2A is a process flow for an example method for autonomous control of a robot arm coupled to a transducer enabling automated echocardiogram scanning.

FIG. 2B shows an architecture of the automated scanning system illustrating three asynchronous components, with their inputs on the left side and outputs on the right.

FIG. 2C is an example neural network with an echocardiogram input and six (6) values representing location and orientation of tool control point (TCP) as output.

FIG. 2D is a schematic of one embodiment of a system illustrating the collaboration among its various components.

FIGS. 3A and 3B show images of the alignment results of an example pair of echocardiography images in the long-axis view without the use of robot arm-based tracking information (FIG. 3A) and with the use of robot arm-based tracking information (FIG. 3B). One of the volumes and the corresponding LV annotation are rendered in green color, whereas the other volume and annotation are in purple.

FIGS. 4A and 4B show the alignment results of an example pair of echocardiography images in the short-axis view without the use of robot arm-based tracking information (FIG. 4A) and with the use of robot arm-based tracking information (FIG. 4B). The first volume and the corresponding LV annotation are rendered in green color, whereas the second volume and the annotation are rendered in purple.

FIG. 5 is a comparative plot of the Dice coefficient between the first scan and other scans at end-diastole with and without robot arm-based alignment of images acquired from two volunteer participants. A total of five scans from volunteer 1 (V1) and 11 scans from volunteer 2 (V2) were used.

FIG. 6 is an example planar views of the 3D echocardiography scan generated by one embodiment of a robot scanning system.

FIG. 7A is boxplot showing the range of force applied along the direction of the probe onto the participant during the echocardiography scanning. The range values demonstrate that no more than 6N of force was applied during the scanning.

FIG. 7B is a boxplot illustrating the distribution of forces applied in the lateral x direction of the transducer by the participants during echocardiography scanning. Despite some values exceeding 10N, it is important to note that these forces are generated by the participants themselves and do not raise safety concerns.

FIG. 7C is a boxplot illustrating the distribution of forces applied in the lateral y direction of the transducer by the participants during echocardiography scanning. Despite some values exceeding 6N, it is important to note that these forces are generated by the participants themselves and do not raise safety concerns.

FIG. 8 are example graphs showing force measurements in the directions of the probe (Force-Z) as well as lateral directions (Force-X and Force-Y) over time. The vertical red lines show the start times of echocardiography scanning.

FIG. 9 is an example flowchart for training of a neural network navigation model.

FIG. 10 is an example flowchart of autonomous robot-arm assisted 2D echocardiography.

### DETAILED DESCRIPTION OF THE EMBODIMENTS

### General Overview

Real-time three-dimensional (3D) echocardiography is often preferred over 2D echocardiography because it allows for scanning the heart in 3D and significantly improves the field of view, but suffers from occasional signal dropout when the angle of the incident is small. One solution is to scan the heart from different positions. Apical and parasternal views are two of the commonly used views in clinics to scan the heart to get different parts imaged adequately. However, scanning different positions requires tracking the ultrasound transducer's position in real-time and combining multiple scans.

Commercial echocardiography scanning systems are not capable of tracking the positions of the transducers during scanning. Unlike magnetic resonance imaging or computed tomography systems, there are no built-in mechanisms available in echocardiography scanners to cross-reference scans acquired from different positions, and cardiologists look at the scans acquired from apical and parasternal views separately.

External trackers could be used to obtain the position and orientation information of the ultrasound transducers to cross-reference apical and parasternal views. Although optical or electromagnetic tracking systems could be used to solve the same problem, they suffer from some limitations. For instance, optical tracking systems require line-of-sight between the markers and tracking cameras, and the system becomes useless when a sonographer accidentally blocks the view of the markers from the cameras. Electromagnetic systems are affected by other ferromagnetic materials in the vicinity of the scanning system, and setting up a scanning room without any ferromagnetic material is quite challenging.

In view of the foregoing, disclosed examples provide for a human-robot collaborative system to perform the scanning and tracking the transducer positions in 3D. As used herein, a “cobot” is a collaborative robot which is a robot used with direct human interaction, preferably in a shared space where the human and the robot are in close proximity. A “robot” is a machine, preferably one which is computer programmed, which is configured to carry out a series of physical actions automatically. A robot may be guided by human intervention while carrying out its designed tasks. A robot which can perform without any human intervention is an “autonomous” robot, while one that functions with some degree of human intervention may be a “semi-autonomous” robot.

Accordingly, disclosed examples use the term “cobot” in the context of the fusion of multiple echocardiography views. More generally, collaborative robot arms and cobots are used to provide accurate real-time tracking of the ultra-sound transducer, facilitating the option of aligning and combining multiple scans.

### Example System

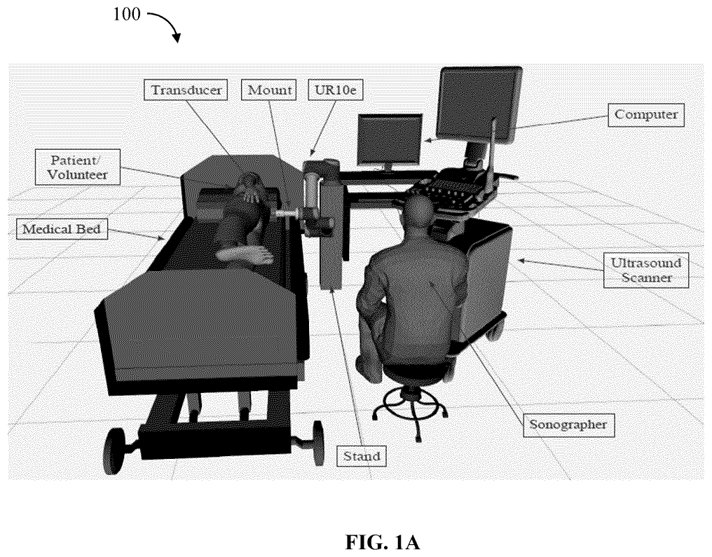

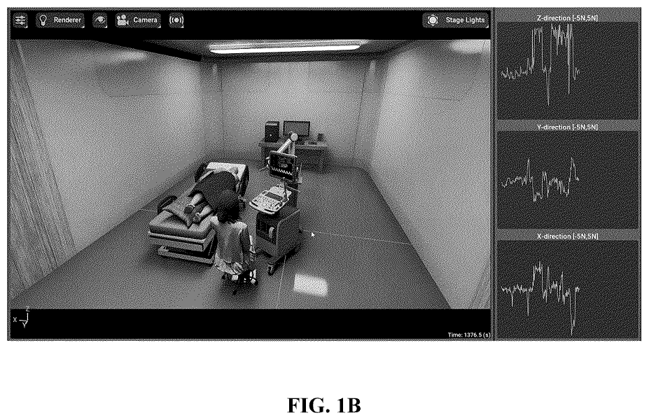

FIG. 1 exemplifies a system 100 for a robot scanning and tracking of transducer positions in 3D. The system 100 allows scanning the cardiac structures from different positions, which are aligned using the tracking information obtained using the robot arm. The resulting ultrasound volumes/images can be aligned using the robot's translation and orientation information.

Generally, an embodiment of the system includes a transducer scanner coupled to a robot arm. In some examples, the system includes a UR10e™ arm (Universal Robots, Odense, Denmark) and a Philips EPIQ™ 7C scanner (Philips Healthcare, Eindhoven, Netherlands). In other examples, the transducer scanner may be either a 2D or 3D scanner.

The robot arm moves the transducer to the desired scanning locations while tracking the positions of the transducer during scanning. In at least one example, the ultrasound transducer is mounted to the robot arm in any suitable manner, preferably using a custom mount, which may be produced by additive manufacturing, for example. The surface definitions of the ultrasound transducer may be obtained using a laser scanner for the precise design of the mount.

The robot arm is controlled using a suitable controller for controlling the robot arm movement. The controller can comprise a Real-Time Data Exchange (RTDE) interface (Universal Robots, Odense, Denmark), which allows for communicating with the arm at 500 Hz.

The robot arm may be, itself, mounted on a stationary pedestal. The robot arm system is operably connected to a computer for real-time control and data transfer, for example, a wired Ethernet connection. In at least one example, Python™ is used as the primary programming language to implement the software components of the system, although other languages may also be suitable.

In some examples, a commercially available image analysis module (e.g., a 3DSlicer1™ module) is used for processing images captured by the transducer, computing transformations, and displaying the final results.

In use, the scanning 2D or 3D views may comprise apical, right ventricular (RV)-focused, and/or parasternal echocardiography views. Preferably, a multi-beat acquisition option is used for scanning, which involves four consecutive cardiac beats being used in each scan to obtain sub-volumes of the heart. The sub-volumes are combined by the echocardiography machine to generate the final scan. Upon completion of the scanning session, the data is exported to a Cartesian coordinate system for post-processing which includes the geometric transformation of each volume sequence to align them in a common coordinate system.

The disclosed robot-arm-based multi-view echocardiography scanning system does not suffer from some of the limitations posed by previous optical or electromagnetic tracking-based systems. Optical tracking systems require a line of sight between the optical markers and cameras, whereas the electromagnetic system requires the surrounding environment to be free of any ferro-magnetic materials for accurate tracking.

Although a passive measurement arm could be used in place of a robot arm, the robot arm offers additional advantages over a passive measurement arm. These advantages include an option to teleoperate the robot arm to reduce sonographer strain during scanning and to implement a semi-automated or fully automated system.

As disclosed herein, in preferred embodiments, the arm comprises force and torque sensors. A UR10e™ arm has integrated force and torque sensors. The force mode allows for investigating and estimating the required minimum force to obtain optimal-quality images. This enhances patient comfort since the robot arm is programmed to not exceed the force required for scanning. Preferably, the system comprises a safety feature which permits a patient to simply push the arm away if they need to get off the bed during the scanning.

At the time of the scanning, the robot movement is restricted only in the direction of the ultrasound transducer, which allows for keeping the transducer in the correct orientation and preventing it from drifting away from the desired scanning location.

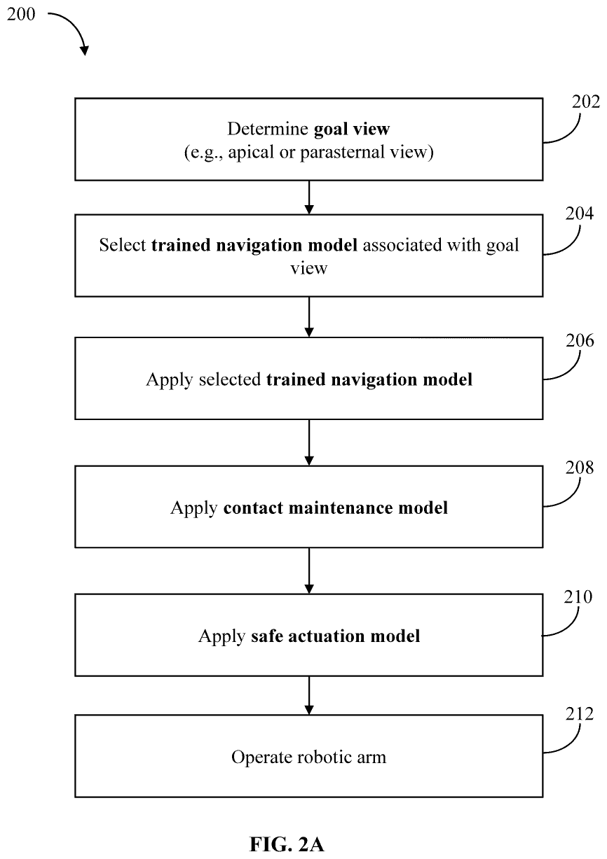

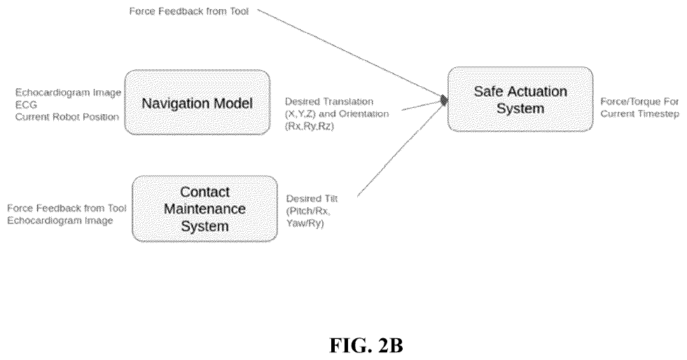

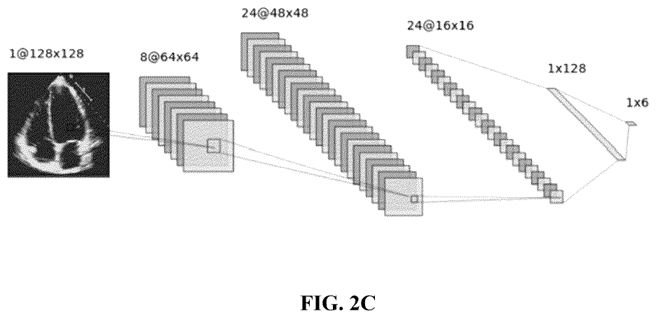

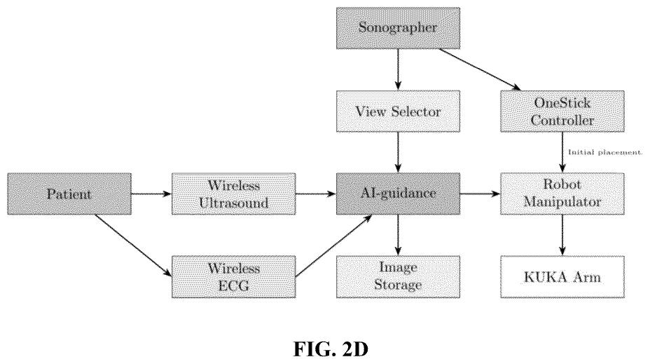

FIG. 2D shows a schematic of a preferred embodiment of a semi-automated system, illustrating the collaboration among its various components. As shown, the sonographer initiates the process by placing the transducer using a controller, such as a OneStick™ or other suitable input controller that allows robot movements with 6 degrees of freedom, and employing a view selector program to designate specific views for scanning. A guidance program, which may comprise a trained AI model, utilizes real-time data from the scanner to compute the optimal positions and orientations for the transducer, aligning with the selected standard views. Subsequently, the robot manipulator physically adjusts the robot arm to these target positions, where images are captured and stored in the workstation.

### Method of Semi-Autonomous Control

In at least one example, the robot arm is moved semi-automatically, with the sonographer guiding the robot arm, mostly manually, to a desired location.

In this example mode, the robot operates to initially position itself to scan the desired view of the heart autonomously. This can occur through a button command on a controller (e.g., a OneStick wireless controller) or a keystroke command from a keyboard, or any other suitable user interface. Subsequently, the sonographer uses the controller to guide the robot arm to an optimal position and orientation for probe placement, which may vary among participants and require precise adjustments for each individual.

To ensure effective use of the robot echocardiography scanning system, it is preferred to achieve adequate force application during scanning. Excessive force can cause discomfort to patients, whereas insufficient force could compromise image quality. Robot arms with force and torque sensors allow the use of force feedback to regulate the pressure exerted by the transducer on the patient. This feature also facilitates free-floating operation of the robot arm when not actively scanning to improve safety protocols.

During the navigational phase, the robot arm's movement is controlled by force values to ensure safety, stopping if it encounters the participant or any obstacles. Once the optimal position and orientation of the ultrasound transducer are determined, the movement direction of the robot arm is fixed to maintain a consistent orientation throughout the scanning process for that particular view, preventing image artifacts. A constant contact force, for example approximately 5N, is applied in the ultrasound transducer direction during scanning to maintain contact between the transducer and the participant's skin, minimizing air gaps that could degrade image quality. This consistent force also improves safety by allowing participants to push the arm away with a small force exceeding 5 N if necessary.

More generally, the system implements a force mode option to apply the desired pressure to keep the transducer in contact with the patient. In some examples, the force mode is implemented using a Real-Time Data Exchange (RTDE) library. The force mode can also used for retracting the transducer upon completion of each scan and making any translational movement in the x, y, z-coordinate directions, and rotational movement along all three axes for positioning the transducer in a desired position and orientation.

In some examples, as noted, a wireless keyboard can be used for controlling different actions related to the movement of the robot arm using the force mode. Keyboard control is also used to move the robot arm automatically to predefined poses corresponding to apical and parasternal scans and may be used in conjunction with the controller (e.g., OneStick). For example, a keystroke can be used to move the robot arm to an initial position, in close proximity to the selected chest area, but not in contact with the patient. The sonographer can then use the controller move the robot arm to the precise desired location, in contact with the patient.

Thus, a robot ultrasound scanning system can mitigate musculoskeletal strain for sonographers. Research indicates that sonographers apply forces as high as 36N in certain cases. Extensive manual scanning has been identified as one of the leading causes of work-related musculoskeletal injuries among sonographers. Using a lightweight wireless controller to navigate the robot arm, the robot echocardiography scanning system presents a compelling solution to alleviate the strain on sonographers during scanning procedures.

### Method of Autonomous or Semi-Autonomous Control

FIG. 2A shows a process flow for an example method 200 for autonomous or semi-autonomous control of the robot arm in system 100.

At 202, a desired goal view is determined. For example, this can be the apical or parasternal view for an echocardiogram. This may be determined automatically and updated constantly based on recent ultrasound image data, or via user input.

At 204, a trained navigation model associated with that the view is selected, which can be done automatically. For instance, the system memory may store separate trained navigation models, including a trained navigation model for the apical view and a trained navigation model for the parasternal view. Each navigation model can guide the robot arm to the appropriate goal view, depending on whether 2D or 3D scanning is desired. In at least one example, the trained navigation models are trained machine learning models, such as a neural network.

At 206, the selected trained navigation model is applied. The trained navigation model is applied to determine the desired translation position coordinates (x,y,z) and orientation coordinates (Rx, Ry, Rz) to move the transducer tip to a target scanning position associated with the goal view.

In some examples, as shown in FIG. 2B, the trained navigation model receives inputs that include: (i) echocardiogram image generated by the transducer at its current position; (ii) coordinates (e.g., position and orientation) of the robot at its current position; and (iii) electrocardiogram (ECG) data, obtained for example from ECG probes attached to the user.

The output of the navigation model is then the desired position and orientation. Accordingly, this can generate values representing the delta between the current robot tool control point (TCP) orientation and position and the goal orientation and position.

At 208, a contact maintenance model is applied. At a general level, the purpose of the contact maintenance model is to determine the tilt (pitch/Rx and yaw/Ry) required to maintain desired contact with body and ideal vision of the heart. During movement, there is a constant 5N force applied in the direction of the tool (i.e., towards the direction the camera is facing) to maintain contact with the body. As the surface may vary, this control block is responsible for tilting the camera to ensure it remains perpendicular to the surface. As shown in FIG. 2B, the output of the contact maintenance model is two numbers representing the tilting required to maintain contact with the body which are then fed into the safe actuation model (explained below). This can be approached using one of the following methods, or a combination of both:

Example Approach 1: This example approach is based on Norm of the Force Feedback. In this approach, the force feedback is used to determine whether the tool is making sufficient contact with the body. In this case, the force feedback is entirely in the opposite direction of the tool/transducer. Thus, if the direction vector of the force feedback is not entirely in line with the tool, the tool must be readjusted. By using the force-feedback as input to determine which axis needs to tilt, the system can autonomously output the desired tilt in two axes, which will be passed onto the safe actuation model.

Example Approach 2: This is based on using an echo image. Using the echo image as an input into an algorithm can be used to determine the tilt required to keep the heart in view. This can involve hand-crafted algorithms based on human supplied heuristics, or machine learning models, or a combination of the two.

At 210, the safe actuation model is applied. The safe actuation model receives the desired location and orientation from the navigation model and the contact maintenance model, and uses a classical control algorithm to change orientation and position of the robot arm safely.

In a preferred embodiment, the output of the navigation model (act 206) comprises six values representing the desired translational and orientation movement. The output of the contact maintenance model (act 208) is two values representing the pitch and yaw required to keep the transducer perpendicular to the body. The safe actuation model modulates these high-level directions into safe actuations to limit the total force outputted to the selected values, for example a force maximum of 5N, and 3 Nm of torque.

Tilt values are outputted by both the navigation model and the contact maintenance model. In some embodiments, these outputs can be combined to determine the optimal tilt. Alternatively, the navigation model's tilt can be discarded in favor of the contact maintenance model, however in alternative embodiments, the outputs may also be averaged, equally or in a weighted manner.

The safe actuation model treats force and torque separately but follows the same process for both. First, based on the goal movement the system creates a desired direction vector. Then it normalizes the current goal direction vector such that the output is a magnitude one vector in the direction of the desired movement. It then multiplies this by 5N for translation and 3 Nm for rotation. The system then passes the desired force and torques into a force control method to limit the total force outputted.

Limiting the total force outputted is achieved by leveraging the force control methods available for industrial robots, or by implementing a simple PID controller, or another method, to control the robot while scaling the speed of the robot/tool based on the feedback of force sensor(s). These approaches enable users to instruct the robot to move with a pre-defined force/torque in each direction.

At 212, the robot arm is controlled based on the output of the safe actuation model. This can involve the robot arm controller receiving the force and torque to apply for the current time stamp, from the safe actuation system.

### Training of Navigation Model

In some examples, separate models are trained for different views. For instance, this may include a training a separate apical navigation model and parasternal navigation model. In either cases, the models can be trained in various suitable manners.

### (i.) Example Training Methodology 1

In at least one example, the navigation model comprises a neural network (see FIG. 2C) trained using an input comprising 2D slices from 3D volume. The output is then the delta between current 2D slice's robot position and the robot position of the goal 2D slice.

In this example, the training data set is generated from 3D volumes which are captured by a sonographer controlling a robot arm. The sonographer manipulates the arm by looking at 2D echocardiogram images in real time. Once the desired view is achieved, the sonographer switches to a 3D acquisition mode to acquire volumes. The system records the robot TCP position/orientation, the 2D echocardiogram images, the ECG, timestamps, and the 3D acquired volumes.

The model is then trained on 2D slices from the acquired 3D volumes. Arbitrary 2D slices can be associated with robot positions by mapping the difference in orientation/translation between the initial 2D slice and the current slice, to the robot's transformation space.

To train the supervised learning model, the inputs are random 2D echocardiogram image slices and six (6) values representing the current robot rotation and location. The desired output is then generated by finding the difference between, (i) the robot position of each random position, and (ii) the robot position of the goal slice. With this training approach a copious amount of training data can be generated from relatively limited amount of scans. The limitation is that the model is limited to navigating when it is “close” to the goal state.

At inference time, the system uses the input ECG data to determine which point in the cardiac cycle the current echocardiogram image is captured at. This will determine whether the image should be used as input to the navigation model, or discarded. Since the systems work asynchronously, if inputs are discarded the overall system will continue to navigate towards the latest identified goal position and orientation.

### (ii.) Example Training Methodology 2

In another example, the machine learning model is trained with a video frame (at time t) as input, and an expert sonographer's movement (at time t+1) as output.

A dataset is constructed of echocardiogram scans where an expert sonographer is manually controlling the robot (via a controller or by hand). The timestamped echocardiogram images are recorded, alongside timestamped positions of the robot, and timestamped ECG signals.

The machine learning model is then trained with the video frames as the input, and the resulting expert sonographer's movement (immediately following the current video frame) as the output.

The model can be a neural network (e.g., a CNN), or may also include a temporal-based component as well (a vision-based transformer, RNN, etc.) (e.g., FIG. 2C)

### (iii.) Example Training Methodology 3

In still another example, the neural network is trained with arbitrary echocardiogram video frames as input, and the delta between current frame's robot TCP position/orientation and the robot TCP position/orientation during acquisition as the output.

As in example 2, a dataset is constructed of echocardiogram scans where an expert sonographer is manually controlling the robot (via a controller or by hand). The timestamped echocardiogram images are recorded, alongside timestamped positions of the robot, and timestamped ECG signals.

In this approach, single frames of the echocardiogram are used as input, along with the ECG, and robot position, and then the delta between the current robot position and the robot position associated with the next goal state (e.g., apical standard, parasternal) are used as the output.

Unlike example 2, the model does not rely on the expert's fine movements, and instead only relies on the expert's final position when acquiring an image.

### (iv.) Example Training Methodology 4

In still another example, the method trains (or use off the shelf) 2D images to complete 3D reconstruction. Alternatively, 3D volumes are used to completely reconstruct the entire heart volume. This then follows a similar process as example 1.

Unlike example 1, this method allows obtaining arbitrary slices from any perspective of the 3D volume, however these 2D slices may not be accurate since they would be generated by an algorithm rather than based on the true anatomy of a heart.

### (v.) Example Training Methodology 5

In a further example, a foundational model is leveraged, whether LLM based, RL-based, or another approach, to perform this task. A foundational model is a model pre-trained on a large corpus of data, which could be related, partially related, or unrelated to the current task. This may consist of a separate model to interpret the echocardiogram images first, and passing in the interpretation (which could be a latent space representation, or just words for an LLM) to a finetuned foundational model, and receive the output to direct the robot to a goal state. It may also consist of directly passing the input into the finetuned foundational model and retrieving the results.

### (vi.) Example Training Methodology 6

In still yet a further example, reinforcement learning is used to train an agent which can determine a path to the goal state. The reinforcement model can be trained on real world data, or in a simulation, or both. It could be a model-free method, or a model-based method. It may leverage on-policy or off-policy learning, or a combination of the two, and may or may not involve neural networks. To train these models, the reward can be based on how close the robot position is to the goal state. Additional rewards may also take into account other factors, such as increasingly negative rewards depending on how many timesteps the input image resembles bone (to disincentivize staying on a rib). For an in depth example of a potential reinforcement learning approach, see option 8.

### (vii.) Example Training Methodology 7

In some examples, an adequate amount of training happens prior to the system being deployed, however there is value in continuously improving the system. Online learning here refers to the ability of the model to continuously improve even after it is deployed. This could include limiting the online learning to only use data collected from the same patient as is currently being scanned, similar patients, or some other subset of data, or may enable learning from all data the system collects while being used. The selection of what data is used may be done automatically, or via human selection.

### (viii.) Example Training Methodology 8

Rather than relying on one fully connected model to learn the navigation task end-to-end, multiple modular models can be used.

### Example Components

A vision model encodes/decodes an arbitrary echo image into a latent space, and be able to reconstruct the original, for example using a variational autoencoder (VAE), which can be used to reconstruct the images from latent space vectors.

Memory model is trained to predict future latent vectors based on the current latent vector, and the subsequent action.

### Controller Model Determines the Optimal Action Based on the Expected Reward

The control model is kept as small as possible, by keeping the majority of complexity in the “world model” made up of a vision and memory block. Separating the vision model from the memory and control can allow making use of other data sources. For example, feeding in arbitrary frames of an echo scans can pre-train the vision model. Then feeding in arbitrary videos of echo scans to pre-train the vision and memory model can build an understanding of a beating heart with zero movement of the transducer. Finally, videos of complete scans can be used to complete training of the overall system.

Embodiments can use neural networks, or other approaches. The approach also offers improved interpretability, as the vision and memory blocks can be used to build simulations. This allow experts to visually verify the system's world model is anatomically correct and will enable improvements based on this human feedback.

### Image Scan Alignment

Once the images are obtained from both the apical and parasternal views (e.g., via method 200 in FIG. 2A), they may be aligned and combined as follows.

### (i.) Image Transformations

The real-time position information of the robot arm is obtained using the Noetic version of the Robot Operating System (ROS) 2 running on the laptop. The tool position of the system is configured to represent the tip of the ultrasound transducer, which allows for obtaining the position and orientation of the transducer directly using the tf module without further computations. However, an additional transformation, Transducer, to account for any coordinate changes between the tool and the echocardiography image. The final geometric transformation, Tvol,n, required for registering the nth scan from a participant, is computed as follows.

$${T}_{vol , n} = {T}_{transducer} \times {T}_{tool , n} \qquad ( 1 )$$

where T_{tool,n} is the tool transformation obtained using tf module during the n^{th} scan. In some examples, the data obtained through the ROS during the scanning session is saved as rosbag files on the laptop. The time information on the laptop and echocardiography scanner is synchronized at the beginning of the session, which allows for using the time stamp information available on the Digital Imaging and Communications in Medicine (DICOM) header of each echocardiography scan to identify the corresponding robot pose.

### (ii.) Left Ventricle Annotations

The overlap between the left ventricular (LV) regions is measured to assess the accuracy of different scans' alignments. The overlap between LV regions from two different scans will be higher when the scans are aligned, and the value will be lower when they are not aligned well.

In order to get the LV regions, the scans are delineated by a sonographer using commercially available TomTec Arena™ (TomTec Imaging Systems, Unterschleissheim, Germany) software. The LV annotations are performed on the echocardiography images exported directly from the scanners. Therefore, the annotations are in the original image coordinate system. Each LV annotation is transformed using the same transformation applied to the corresponding echocardiography image before computing the overlap measure.

### (iii.) Dice Similarity Coefficient

In at least one example, the overlap between each pair of annotations is computed using a Dice similarity metric (DM). The DM between the i^{th} and j^{th} annotations, i and j, is computed in 3D as follows in Equation (2):

$$DM ( {V}_{i} , {V}_{j} ) = \frac{2 {V}_{ij}}{{V}_{i} + {V}_{j}} \qquad ( 2 )$$

where Vij is the intersecting volumetric region between Vi and Vj. In this case, DM=1 means a complete match, and DM=0 means no overlap between the annotations. The SimpleITK Python module is used for reading and processing the LV annotations as well as computing the DM values.

### Integration of Robot Technology for Combining Multiple Views in Three-Dimensional Echocardiography

In some embodiments of a robot-assisted echocardiography system, the system is configured to track the position and orientation of the transducer in three-dimensional space. This positional information can be utilized to register, align, and fuse multiple echocardiographic acquisitions, whether obtained as two-dimensional images or as three-dimensional volumes. Such fusion enables expansion of the effective field-of-view and improvement of overall image quality.

### (i.) Scanning Tests.

Scans were acquired from two healthy volunteer participants and were processed and used for the evaluation of one embodiment of the system. A total of 16 echocardiography scans were used. The image dimensions were 272×176×208 and the resolutions were 0.775 mm×1.209 mm×0.778 mm in x, y and z-coordinate directions, respectively.

### (ii.) Visual Assessment.

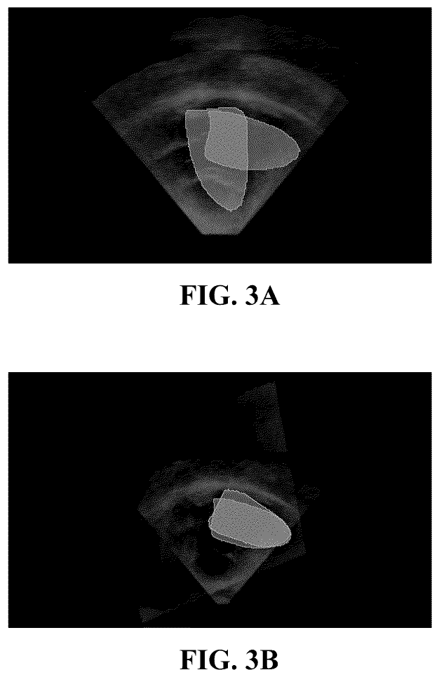

The results showing the alignment of volumes and the corresponding LV annotations were evaluated visually using 3DSlicer™ software. Example results visualized in the long-axis view at the end-diastolic cardiac phase are given in FIGS. 3A and 3B, where FIG. 3A shows the alignment results without the robot arm. FIG. 3B shows the alignments of echocardiography images and the corresponding annotations when the robot arm information is used for aligning the images.

The results presented in FIG. 3 demonstrate the benefits of using a robot arm for multi-view echocardiography fusion, as a better alignment is achieved when the images are transformed using the tracking information.

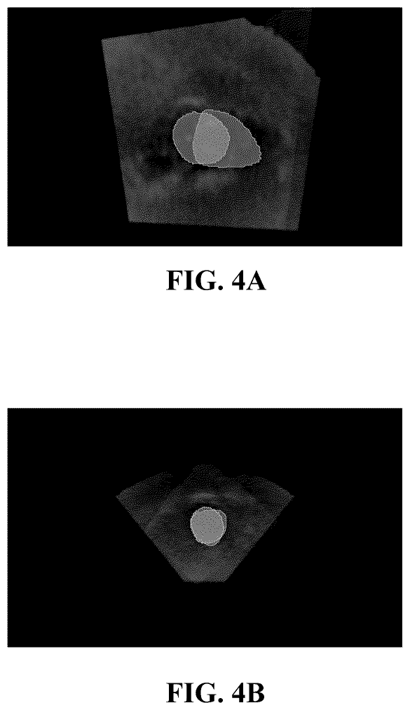

The corresponding results in the short-axis view are given in FIG. 4. The results in FIGS. 4A and 4B show that the alignment accuracy was significantly improved when the tracking information from the robot arm was utilized.

### (iii.) Quantitative Evaluation.

Quantitative evaluations were performed by computing the Dice coefficients between the pair of annotations corresponding to the first scan and each other scan from each volunteer. The Dice coefficient values were calculated at the end-diastolic cardiac phase with and without the application of the geometric transformations computed for each volume.

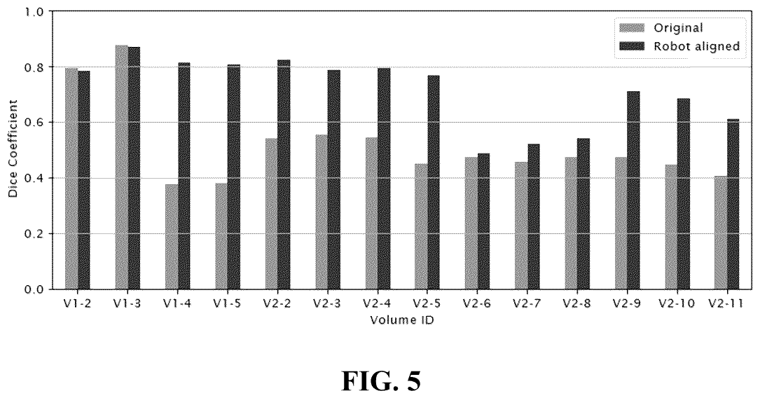

Bar plots in FIG. 5 show the results for each pair of volumes for volunteer 1 (V1) and volunteer 2 (V2). A total of 5 scans from Volunteer 1 and 11 scans for Volunteer 2 were used in the evaluation.

Overall, a significant improvement in the Dice coefficient was observed when the information from the robot arm was used for the alignment, especially between the first and fourth or fifth scans from Volunteer 1. Similarly, significant improvements in the Dice coefficients were observed between the first and fourth, fifth, ninth, tenth, and eleventh scans from Volunteer 2.

The mean and standard deviation of Dice coefficient values for the overall evaluation is given in Table I. A total of fourteen Dice coefficient values corresponding to the end-diastolic phase were used in the evaluation.

The results show that there was an overall significant improvement in the Dice coefficient when the information from the robot arm was used for computing the alignment of echocardiography images.

TABLE I
Mean and standard deviation dice score values measuring
the overlap of left ventricular volume annotations corresponding
to the first scan and other scans from the same volunteer -
the higher the value, the better the overlap.

| | Dice Coefficient | Mean | Standard deviation |
|---|---|---|---|
| Method | Original | 0.519 | 0.146 |
| | Robot aligned | 0.716 | 0.126 |

### Example Test Results Force Application

### (i.) Data

The images obtained from thirty healthy volunteers (17 males; 13 females) were processed and analyzed to evaluate the effectiveness of the system.

A total of 836 3D echocardiography scans from 30 volunteers were used, with image dimensions measuring 272×176×208 and resolutions of 0.775 mm×1.209 mm×0.778 mm in coordinate directions x, y, and z, respectively.

### (ii.) Results

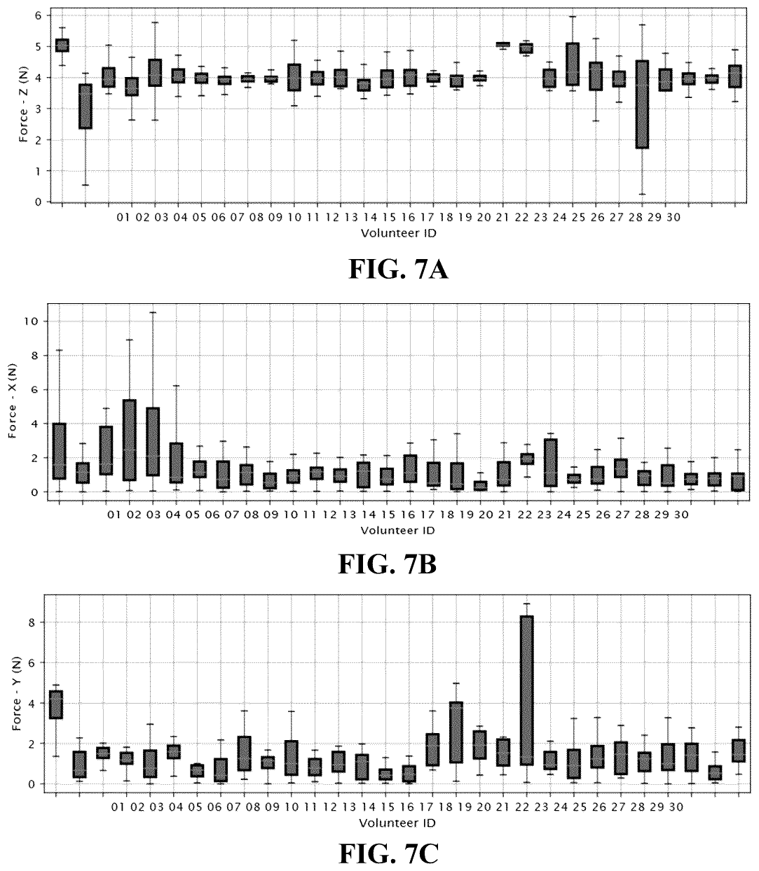

Throughout the scanning procedure, the force values were recorded in the ultrasound transducer direction by the UR10e arm sensors. The force values in the ultrasound transducer direction for the 30 volunteer participants during scanning are shown in the box plot presented in FIG. 7A. The plots demonstrate that only the required amount of force was applied to all participants by the system and never exceeded 6N for any of the participants.

The force values in the lateral directions of the ultrasound transducer were also recorded during the scanning process. In our design, the robot arm does not apply any force in the lateral directions; however, it is restricted to move only in the direction of the transducer to maintain consistent orientation during scanning. Therefore, the force values measured in the lateral directions represent the force applied by the participants to the transducer.

FIGS. 7B and 7C display box plots illustrating the force values in the lateral direction of the transducer for the 30 participants. Although some of these values exceed 6N, they do not raise safety concerns as they represent the force applied by the participant to the robot arm.

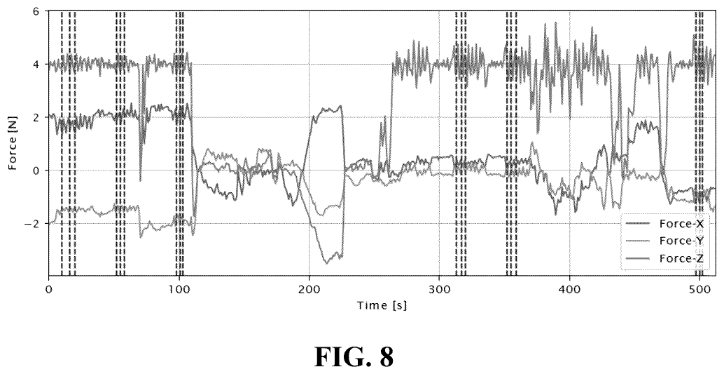

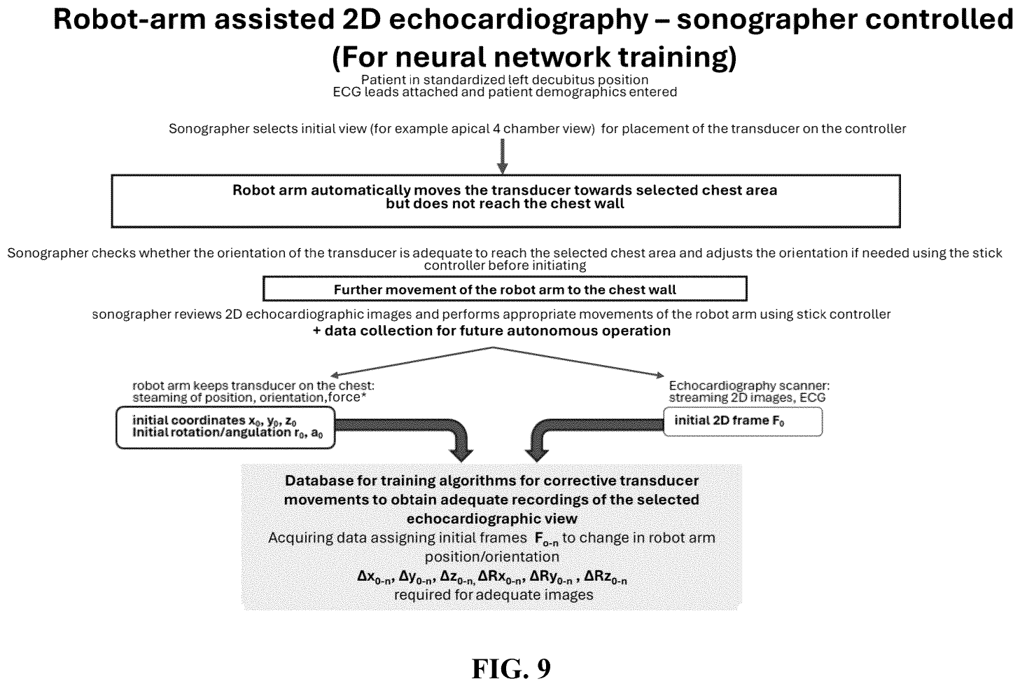

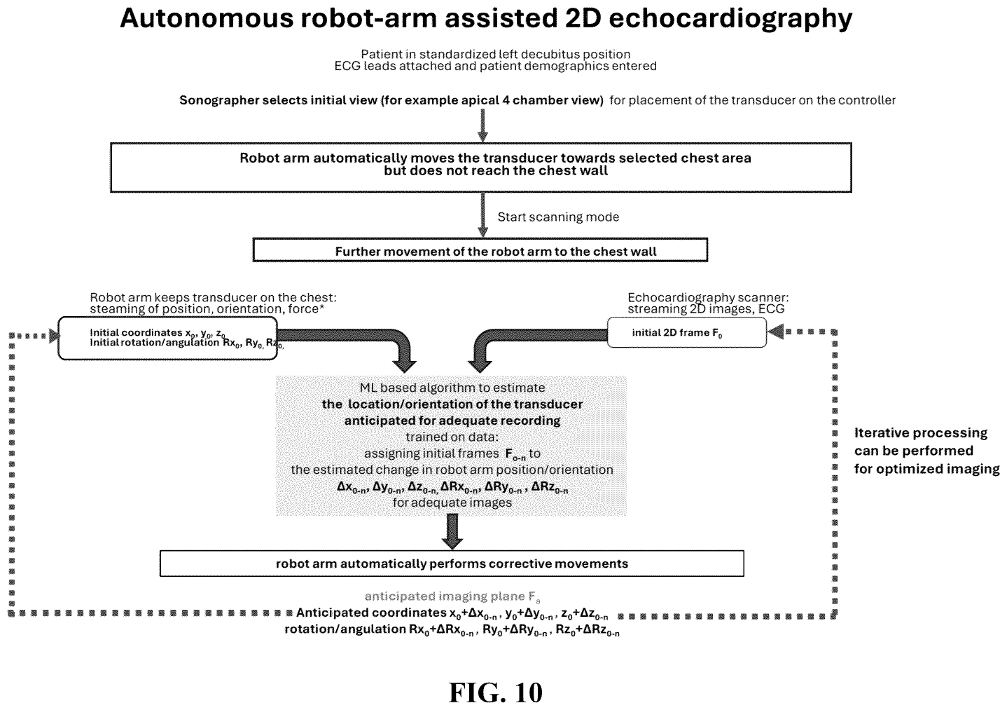

The force values recorded in the x, y, and z directions for a representative participant throughout the scanning process, from the first scan to the last, are depicted in FIG. 8.

The vertical dotted red lines in the figure indicate the times at which the echocardiography scans were acquired. The green curve (Force-z) illustrates the force measurements in the ultrasound transducer direction, while the blue and orange curves represent the force measurements in the lateral directions of the transducer. In particular, the green curve demonstrates that the forces applied in the transducer direction during the process do not exceed 6N, remaining within the comfortable limit of the participant.

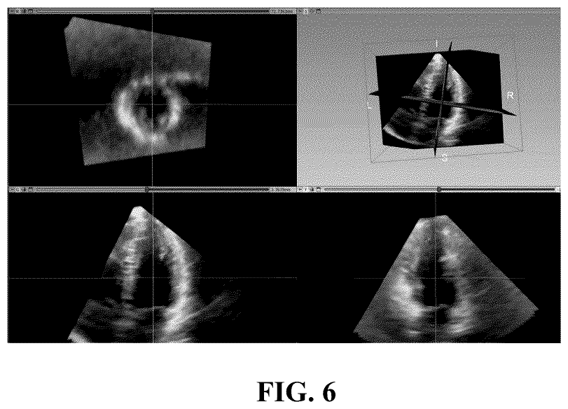

The visual assessment of the echocardiography scans demonstrated that the image quality was not compromised by the small amount of force applied by the scanning system. Planar views of the 3D echocardiography image for a representative example are shown in FIG. 6.

Accordingly, the performance of the system was evaluated with 30 healthy volunteers, revealing that the controlled force operation of the robot arm is an effective approach to scanning human subjects. It allows for applying a desired force well below what is typically exerted during manual scanning without compromising image quality. The average duration between the first and last scans for the volunteers was 16.8±10.3 minutes for a total of 836 3D volumetric sequences acquired from 30 participants.

### I. Interpretation

Various systems or methods have been described to provide an example of an embodiment of the claimed subject matter. No embodiment described limits any claimed subject matter and any claimed subject matter may cover methods or systems that differ from those described below. The claimed subject matter is not limited to systems or methods having all of the features of any one system or method described below or to features common to multiple or all of the apparatuses or methods described below. It is possible that a system or method described is not an embodiment that is recited in any claimed subject matter. Any subject matter disclosed in a system or method described that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

The terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device. As used herein, two or more components are said to be “coupled”, or “connected” where the parts are joined or operate together either directly or indirectly (i.e., through one or more intermediate components), so long as a link occurs. As used herein and in the claims, two or more parts are said to be “directly coupled”, or “directly connected”, where the parts are joined or operate together without intervening intermediate components.

Terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The present invention has been described here by way of example only, while numerous specific details are set forth herein in order to provide a thorough understanding of the exemplary embodiments described herein. However, it will be understood by those of ordinary skill in the art that these embodiments may, in some cases, be practiced without these specific details. In other instances, well-known methods, procedures and components have not been described in detail so as not to obscure the description of the embodiments. Various modification and variations may be made to these exemplary embodiments without departing from the spirit and scope of the invention, which is limited only by the appended claims.

1. A collaborative robot system for ultrasound scanning, comprising:
a robot arm;
an ultrasound transducer coupled to an end of the robot arm;
a controller for controlling movement of the robot arm;
at least one processor configured for:
determining a goal view;
selecting a trained navigation model associated with the goal view;
applying the selected navigation model to generate movement coordinates for translating and orienting the robot arm to the goal view;
applying a contact maintenance model to generate an orientation command for the transducer;
applying a safe actuation model based on the outputs of the navigation model and/or contact maintenance model to generate torque and force outputs to operate the robot arm; and
operating the robot arm based on torque and force outputs generated by the safe actuation model.

2. The system of claim 1 configured for echocardiography scanning.

3. The system of claim 2, wherein the at least one processor comprises an image analysis module configured for aligning images generated from scans from different goal views.

4. The system of claim 1 wherein the robot arm comprises force and/or torque sensors which modulate the navigation model, the contact maintenance model, and/or the safe actuation model to regulate contact of the transducer with a patient.

5. The system of claim 4 wherein the contact maintenance model is configured to maintain a consistent orientation and constant contact force with the patient once the transducer is correctly positioned on the patient for the selected goal view.

6. The system of claim 1 which is a 2D or a 3D system.

7. The system of claim 1 wherein the safe actuation model permits the robot arm to be moved away from the patient at any time by manual force.

8. The system of claim 1, wherein the navigation model is configured to receive an input comprising one or more of (i) an ultrasound image generated by the transducer at its current position; (ii) coordinates of the robot arm at its current position; and (iii) electrocardiogram (ECG) data obtained from ECG probes attached to the patient.

9. A method of controlling a robot arm for ultrasound scanning of a patient, using an ultrasound transducer coupled to an end of the robot arm, comprising the steps of:
determining a goal view;
selecting a trained navigation model associated with the goal view;
applying the selected navigation model to generate movement coordinates for translating and orienting the robot arm to the goal view;
applying a contact maintenance model to generate an orientation command for the transducer;
applying a safe actuation model based on the outputs of the navigation model and/or contact maintenance model to generate torque and force outputs to operate the robot arm; and
operating the robot arm based on torque and force outputs generated by the safe actuation model.

10. The method of claim 9 which is an echocardiography scanning method.

11. The method of claim 9 comprising wherein the steps are repeated for different goal views, and comprising the further step of aligning the resulting different goal view images.

12. The method of claim 9 wherein force and/or torque sensor values are obtained and used to modulate the navigation model, the contact maintenance model, and/or the safe actuation model to regulate contact of the transducer with a patient.

13. The method of claim 9 wherein the contact maintenance model is configured to maintain a consistent orientation with the patient once the transducer is correctly positioned on the patient for the selected goal view.

14. The method of claim 9 wherein the navigation model receives an input comprising one or more of (i) an ultrasound image generated by the transducer at its current position; (ii) coordinates of the robot arm at its current position; and (iii) electrocardiogram (ECG) data obtained from ECG probes attached to the patient.

15. The method of claim 13 wherein the contact maintenance model output overrides the navigation model output, or the contact maintenance model output and the navigation model output are averaged to position the robot arm.

16. The method of claim 9 wherein the safe actuation model permits the robot arm to be moved away from the patient by manual force.
